(19) United States
(12) Patent Application Publication
(10) Pub. No.: US 2022/0401441 A1
(43) Pub. Date: Dec. 22, 2022

(54) PHARMACEUTICAL COMPOSITION COMPRISING SELEXIPAG

(71) Applicant: ACTELION PHARMACEUTICALS LTD, Allschwil (CH)

(72) Inventors: Marc Patrik SCHRADER, III, Allschwil (CH); Alexandra SCHLICKER-SPAIN, Allschwil (CH)

(21) Appl. No.: 17/770,155

(22) PCT Filed: Oct. 22, 2020
(86) PCT No.: PCT/EP2020/079698

§ 371 Date: Apr. 19, 2022

(30) Foreign Application Priority Data
Oct. 23, 2019 (EP) PCT/EP2019/078905

(51) Int. Cl. A61K 31/4965; A61K 9/20; A61K 9/28

(52) U.S. Cl. CPC A61K 31/4965; A61K 9/2018; A61K 9/2059; A61K 9/2054; A61K 9/2013; A61K 9/2866

(57) ABSTRACT

The present invention relates to pharmaceutical compositions comprising 2-{4-[N-(5,6-diphenylpyrazin-2-yl)-N-isopropylamino]butyloxy}-N-(methylsulfonyl)acetamide (selexipag, NS-304, ACT-293987) which are suitable for oral administration (p.o.).

The present invention relates to pharmaceutical compositions comprising 2-{4-[N-(5,6-diphenylpyrazin-2-yl)-N-isopropylamino]butyloxy}-N-(methylsulfonyl)acetamide (selexipag, NS-304, ACT-293987; hereinafter COMPOUND) which are suitable for oral administration (p.o.).

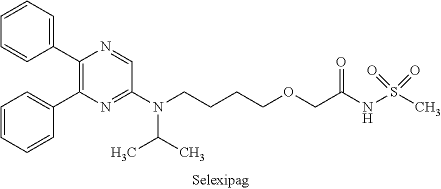 [SMILES: CC(C)N(CCCCOCC(=O)NS(C)(=O)=O)c1cnc(-c2ccccc2)c(-c2ccccc2)n1]

The preparation and the medicinal use of selexipag and its active metabolite 2-(4-((5,6-diphenylpyrazin-2-yl)(isopropyl)amino)butoxy)acetic acid (MRE-269, ACT-333679) is described in WO2002/088084; WO2009/157396; WO2009/107736; WO2009/154246; WO2009/157397; WO2009/157398; WO2010/150865; WO2011/024874; Nakamura et al., Bioorg Med Chem (2007), 15, 7720-7725; Kuwano et al., J Pharmacol Exp Ther (2007), 322(3), 1181-1188; Kuwano et al., J Pharmacol Exp Ther (2008), 326(3), 691-699; O. Sitbon et al., N Engl J Med (2015), 373, 2522-33; Asaki et al., Bioorg Med Chem (2007), 15, 6692-6704; Asaki et al., J. Med. Chem. (2015), 58, 7128-7137. Certain formulations are disclosed in WO2013/024051, WO2014/069401 and WO2018/162527.

Selexipag was shown to be beneficial in the treatment of pulmonary arterial hypertension for adults. In a phase III clinical trial, among patients with pulmonary arterial hypertension, the risk of the primary composite end point of death or a complication related to pulmonary arterial hypertension was significantly lower among patients who received selexipag than among those who received placebo. Selexipag received market approval e.g. in the US and is indicated for the treatment of pulmonary arterial hypertension (PAH, WHO Group I) to delay disease progression and reduce the risk of hospitalization for PAH.

Selexipag is thought to function as a prodrug (while retaining some agonistic activity on the IP receptor on its own) which can exert long-lasting selective IP receptor agonist activity of the active metabolite 2-(4-((5,6-diphenylpyrazin-2-yl)(isopropyl)amino)butoxy) acetic acid in mammals, especially humans. The in vivo metabolism of selexipag effectively may act as a kind of ‘slow-release mechanism’ that potentially both prolongs activity and reduces typical adverse effects associated with high concentrations of PGI2 agonists (Kuwano et al., J Pharmacol Exp Ther (2007), 322(3), 1181-1188).

Adverse effects associated with PGI2 agonists are also addressed by a particular up-titration schedule. The recommended starting dose of oral selexipag for adults is 200 micrograms given twice daily. The dose is then increased in increments of 200 micrograms twice daily, usually at weekly intervals, to the highest tolerated dose up to 1600 micrograms twice daily. If a patient reaches a dose that cannot be tolerated, the dose should be reduced to the previous tolerated dose.

Selexipag is a selective IP-receptor agonist for oral use with proven efficacy and safety in adults with PAH. To date, selexipag is the only IP-receptor agonist approved globally for long-term treatment across WHO FC II-III and primarily in combination with current first-line oral PAH-specific medicines, in adult patients in need of additional therapy because of insufficient disease control. Selexipag represents an important additional treatment option for these patients.

The availability of selexipag, a highly selective IP-receptor agonist for oral use and with demonstrated benefit on PAH disease outcomes in add-on therapy, provides an important rationale to initiate a prostacyclin-pathway therapy at a medically appropriate stage of PAH disease, without major consequences for the patient's lifestyle.

Pediatric PAH is a rare and progressive disorder associated with considerable morbidity and mortality. Current treatment recommendation in the pediatric population includes PDE-inhibitors, ERAs, and inhaled, subcutaneous and intravenous (i.v.) prostacyclin pathway agonists. However, in the absence of randomized controlled clinical trials powered to show efficacy of those therapies in pediatric patients, the treatment algorithm is based on evidence from adult studies. A biocomparison study of adult and paediatric dose strengths of selexipag has been performed (M. Boehler et al, Eur J Drug Metab Pharmacokinet. 2018 February; 43(1):115-120. doi: 10.1007/s13318-017-0424-z).

Patients with hepatic impairment or patients experiencing drug drug interaction with CYP 2C8 inhibitors may also benefit from a dose adaptation to their condition. Preferably, these patients are adult.

Furthermore, patients that do not tolerate the starting dose of 200 mcg may profit from the new dosage.

Therefore, there is a need to develop treatments that may be disease-modifying in pediatric patients or patients with hepatic impairment or patients experiencing drug drug interaction with CYP 2C8 inhibitors, all with PAH and other diseases. Further, a dosing according to the body weight should be allowed. Moreover, treatments should be in a children-friendly form, such as a mini-tablet.

The present invention provides a means for treating pediatric patients with e.g. PAH, which is effective and safe for children with different age classes, such as ≥2 to <6 years of age, ≥6 to <12 years of age and ≥12 to <18 years of age.

Moreover, the present invention provides a means for treating patients with e.g. PAH, suffering from hepatic impairment or experiencing drug drug interaction with CYP 2C8 inhibitors.

1) A first embodiment relates to a pharmaceutical composition comprising the compound of formula (I) in the amount of 80 to 170 mcg

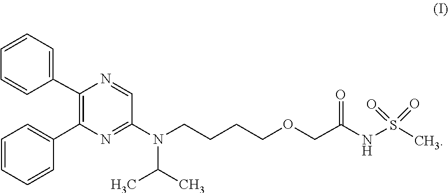 [SMILES: CC(C)N(CCCCOCC(=O)NS(C)(=O)=O)c1cnc(-c2ccccc2)c(-c2ccccc2)n1]

or a pharmaceutically acceptable salt, solvate, hydrate or morphological form thereof.

Thereby, the abbreviation “mcg” stands for microgram, i.e. 1×10⁻⁶ of a gram.

In a preferred embodiment, the pharmaceutical composition comprises the compound of formula (I) in the amount of 80 to 160 mcg, more preferably in the amount of 90 to 110 ug mcg and 140 to 160 mcg, and most preferably in the amount of 93 to 107 mcg and 143 to 157 mcg, e.g. 100 mcg with a tolerance of ±7% and 150 mcg with a tolerance of ±7%. Thereby, the tolerance is applied to a group of 20 tablets.

Preferably, the compound of formula (I), namely 2-{4-[N-(5,6-diphenylpyrazin-2-yl)-N-isopropylamino]butyloxy}-N-(methylsulfonyl)acetamide, in crystalline form, especially in essentially pure crystalline form (preferably in essentially pure crystalline form I or essentially pure crystalline form II as disclosed in WO2010/150865/EP2447254), is used for the preparation of said composition.

2) A further embodiment relates to the composition according to embodiment 1), further comprising one or more selected from the group consisting of:
a) a filler;
b) a disintegrant;
c) a binder; and
d) a lubricant.

Fillers, also referred to as bulking agents or diluents, have several functions, such as diluting the active ingredient within the pharmaceutical composition, they may ensure long-term stabilization or can confer a therapeutic enhancement such as facilitating drug absorption, or enhancing solubility. They may also be useful in the manufacturing process, to aid in the handling of the active substance.

A disintegrant expands when wet, causing the tablet to break apart, for instance in specific segments of the digestion process, releasing the active ingredient for absorption.

Binders hold the ingredients in a tablet together. They ensure that tablets and granules can be formed with required mechanical strength.

Lubricants prevent ingredients from clumping together and from sticking to the tablet punches or capsule filling machine. Lubricants also ensure that tablet formation and ejection can occur with low friction between the solid and die wall.

3) A further embodiment relates to the pharmaceutical composition according to embodiment 2), wherein
the filler, if present, is one or more selected from the group consisting of: D-mannitol, maize starch, lactose, pregelatinized starch, dibasic calcium phosphate dihydrate (CaHPO4.2H2O), microcrystalline cellulose, and maltodextrin; preferred fillers are D-mannitol and maize starch;
the disintegrant, if present, is one or more selected from the group consisting of: low substituted hydroxypropyl cellulose, croscarmellose sodium, sodium starch glycolate, and cross-linked polyvinylpyrrolidone; a preferred disintegrant is low substituted hydroxypropyl cellulose;
the binder, if present, is one or more selected from the group consisting of: hydroxypropyl cellulose, sucrose, gelatin, starch, pregelatinized starch, alginic acid, sodium alginate, methyl cellulose, ethyl cellulose, hydroxy propyl methyl cellulose, polyvinyl pyrrolidinone, calcium carboxymethylcellulose, sodium carboxymethylcellulose, guar gum, clays, ion exchange resins and calcium silicate; a preferred binder is hydroxypropyl cellulose;
the lubricant, if present, is one or more selected from the group consisting of: magnesium stearate, aluminium stearate, calcium stearate, stearic acid, sodium stearyl fumarate, talc, sodium benzoate, glyceryl mono fatty acid, polyethylene glycol, hydrogenated cotton seed oil, castor seed oil, sucrose esters, calcium silicate and silicon dioxide; a preferred lubricant is magnesium stearate.

All listed excipients are commercially obtainable and well known to the person skilled in the art.

A preferred disintegrant is low substituted hydroxypropyl cellulose (L-HPC); IUPAC name: Cellulose, 2-hydroxypropyl ether (low substituted). Particularly preferred is L-HPC with a hydroxypropoxyl content of 7 to 13%, in particular about 10 to 13% (in accordance with USP/NF method).

A preferred binder is hydroxypropyl cellulose (HPC), which is soluble in water due to its large amount of hydroxypropoxyl groups in the cellulose backbone. IUPAC name: Cellulose, 2-hydroxypropyl ether. Particularly preferred is a viscosity (mPa*s) in 2% aqueous solution at 20° C. of 2.0 to 6.0, preferably 2.0 to 5.9, particularly preferred 2.0 to 2.9. The molecular weight (GPC method) is preferably from 34000 to 110000, more preferably from 40000 to 100000, most preferably 40000 (±15%, preferably ±10%).

Reference is made to the extensive literature on the subject for these and other pharmaceutically acceptable excipients and procedures mentioned herein, see for example R. C. Rowe, P. J. Seskey, S. C. Owen, Handbook of Pharmaceutical Excipients, 5th edition (and 6^{th} edition) Pharmaceutical Press 2006; Remington, The Science and Practice of Pharmacy, 21st Edition (2005), Part 5, “Pharmaceutical Manufacturing” [published by Lippincott Williams & Wilkins]. Moreover, reference is made to the brochure of Shin-Etsu Chemical Co., Ltd, Cellulose & Pharmaceutical Excipients Department, 05.8/1000.

4) A further embodiment relates to the pharmaceutical composition according to any one of embodiments 1) to 3), which comprises
D-mannitol and maize starch;
low substituted hydroxypropyl cellulose;
hydroxypropyl cellulose; and
magnesium stearate.

5) A further embodiment relates to the pharmaceutical composition according to any one of embodiments 2) to 4), wherein
(i) the filler is comprised in an amount from 11.5 to 145.0 mg, preferably from 12.0 to 45.0 mg, for example from 12.0 to 35.0 mg;
(ii) the disintegrant is comprised in an amount from 0.6 to 8.5 mg, preferably from 0.6 to 2.5 mg, for example from 0.7 to 2.0 mg;
(iii) the binder is comprised in an amount from 0.5 to 6.5 mg, preferably from 0.5 to 2.0 mg, for example from 0.5 to 1.5 mg; and
(iv) the lubricant is comprised in an amount from 0.2 to 2.5 mg, preferably from 0.2 to 0.7 mg, for example from 0.2 to 0.5 mg.

6) A further embodiment relates to the pharmaceutical composition according to any one of embodiments 1) to 5), which comprises
D-mannitol in an amount from 7.0 to 90.0 mg, preferably from 7.0 to 25.0 mg, for example 7.0 to 20 mg;
maize starch in an amount from 4.5 to 60.0 mg, preferably from 4.5 to 20.0 mg, for example from 4.5 to 15.0 mg;
low substituted hydroxypropyl cellulose in an amount from 0.6 to 9.0 mg, preferably from 0.6 to 3.0 mg, for example from 0.7 to 1.8 mg;
hydroxypropyl cellulose in an amount from 0.5 to 6.5 mg, preferably from 0.5 to 2.0 mg, for example from 0.5 to 1.5 mg; and
magnesium stearate is comprised in an amount from 0.2 to 2.5 mg, preferably from 0.2 to 0.7 mg, for example from 0.2 to 0.5 mg.

7) A further embodiment relates to the pharmaceutical composition according to any one of embodiments 1) to 6), which is in the form of a tablet or a capsule. Preferably, the pharmaceutical composition is in the form of a tablet.

The tablets may vary in shape and be, for example, round, oval, oblong, cylindrical, clover-shaped or any other suitable shape. Preferably, the tablets are round.

Procedures which may be used may be conventional or known in the art or based on such procedures e.g. those described in L. Lachman et al., The Theory and Practice of Industrial Pharmacy, 3rd Ed., 1986; H. Sucker et al., Pharmazeutische Technologie, Thieme, 1991; Hagers Handbuch der pharmazeutischen Praxis, 4th Ed. (Springer Verlag, 1971) and Remington's Pharmaceutical Sciences, 13th Ed., (Mack Publ., Co., 1970) or later editions.

8) A further embodiment relates to the pharmaceutical composition according to embodiment 7), wherein the tablet is coated, the coating material comprising one or more selected from the group consisting of a plasticizer, a film former and a pigment.

Preferably, the tablet is film coated.

Examples for film formers are hypromellose, cellulose acetate phthalate (CAP), acrylate polymers, hydroxypropyl methyl cellulose phthalate (HPMCP) or polyvinyl acetate phthalate (PVAP). It is to be noted that the present list is not limiting. A preferred film former is hypromellose (INN), also known as hydroxypropyl methylcellulose (HPMC).

Plasticizers are added to the polymers used as film forming agents in order to make the polymer pliable and soft, enhancing the flexibility and plasticity of the films. They play a vital role in the formulations like gastro-retentive films, ocular films, transdermal films, buccal films, oro-dispersible films and are added to these products to reduce the glass transition temperature facilitating the thermal stability of the drug and other ingredients.

Preferably, the plasticizer is a hydrophilic plasticizer. Examples for hydrophilic plasticizer are glycerine, polyethylene glycols, polyethylene glycol monomethyl ether, propylene glycol, sorbitol sorbitan solution and triethyl citrate. Preferred is propylene glycol.

A glidant is a substance to be added to improve the powder flow and to reduce the friction or cohesion between particles. Common examples are magnesium stearate, Aerosil (colloidal silicon dioxide), starch and talc. Preferred concentrations of the glidant is 5-10%.

Reference is made to Aulton's Pharmaceutics (The Design and Manufacture of Medicines), 5th edition (editors: Kevin Taylor Michael Aulton), Elsevier.

A preferable coating method used herein is aquatic coating.

Preferred pigments are titanium dioxide, or iron dioxide in any colour.

Additionally, polishing agents may be applied, such as carnauba wax, beeswax or paraffin. Carnauba wax is preferred.

For avoidance of any doubt, it is well understood that pharmaceutical compositions as defined in embodiment 1) to 9) may additionally comprise further conventional ingredients and/or additives, which may be used alone or in combination.

The preferred excipients are specified in the following table, they are all compendial:

TABLE 1

| Excipient | Function |
|---|---|
| D-Mannitol | filler/diluent |
| Maize starch | filler/diluent |
| Low substituted hydroxypropylcellulose | disintegrant |
| Hydroxypropylcellulose | binder |
| Magnesium stearate | lubricant |
| Hypromellose | film former |
| Propylene glycol | plasticizer |
| Titanium dioxide | pigment |
| Iron oxide of any color | pigment |
| Carnauba wax | polish |
| Purified water | solvent |

A further preferred excipient is talc, which functions as glidant.

All excipients comply with European Pharmacopeia, United States Pharmacopeia and Japanese Pharmacopeia.

9) A further embodiment relates to the pharmaceutical composition according to any embodiment from 7) to 9), wherein the tablet is a mini-tablet with a diameter of 1.5 to 4 mm, preferably 2.5 to 4 mm, more preferably 2.7 to 3.5 mm, most preferably 3 mm±0.3 mm.

The pharmaceutical composition according to the preceding embodiments, which is preferably a mini-tablet, has a weight of 12 to less than 50 mg, preferably of 12 to 47 mg.

The pharmaceutical composition according to the preceding embodiments is considered “stable”, if during a certain period of time 70%, preferably 80%, more preferably 90% and most preferably 95% of the initial content of compound of formula I, or pharmaceutically acceptable salt, solvate, hydrate or morphological form thereof, is maintained over said period of time.

The stability of the pharmaceutical composition may be tested in conventional manner, e.g. by measurement of compound of formula I and its degradation products, dissolution, friability, disintegration time, appearance and/or microscopy, e.g. after storage at 25° C. and 60% relative humidity, 30° C. and 75% relative humidity and/or storage at 40° C. and 75% relative humidity for defined periods of time.

Preferably, the solid compositions of this invention will be stable for at least 6 or 12 months when kept at a temperature of 5 to 50° C. More preferably, they will be stable for at least 6 or 12 months when kept at a temperature of 15 to 45° C. Most preferred, they will be stable for at least 12 or 36 months when kept at a temperature of 25 to 40° C. In a more preferred embodiment, the pharmaceutical compositions are stable over a certain period of time such as 1 year, and preferably 2 years. More preferably, the pharmaceutical compositions are stable for 3 to 5 years.

In a preferred embodiment, the mini-tablet according to the invention exhibits particular stability.

The term “pharmaceutical composition” is interchangeable with the term “formulation”, or “composition”.

Whenever the word “between” or “to” is used to describe a numerical range, it is to be understood that the end points of the indicated range are explicitly disclosed and included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

10) The pharmaceutical composition according to the preceding embodiments may be used as a medicament, preferably for oral administration.

11) The pharmaceutical composition according to the preceding embodiments may be used as a pediatric medicament. Preferably, the pediatric patients are from 2 years to <18 years old.

12) The pharmaceutical composition according to the preceding embodiments may further be used in patients with hepatic impairment or patients experiencing drug drug interaction with CYP 2C8 inhibitors such as clopidogrel.

The pharmaceutical composition may also be used for patients which do not tolerate the starting dose of 200 mcg.

13) The pharmaceutical composition according to the preceding embodiments is suitable for use in the prevention and/or treatment of ulcer, digital ulcer, diabetic gangrene, diabetic foot ulcer, pressure ulcer (bedsore), hypertension, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance (e.g., chronic arterial occlusion, intermittent claudication, peripheral embolism, vibration syndrome, Raynaud's disease), connective tissue disease (e.g., systemic lupus erythematosus, scleroderma, mixed connective tissue disease, vasculitic syndrome), reocclusion/restenosis after percutaneous transluminal coronary angioplasty (PTCA), arteriosclerosis, thrombosis (e.g., acute-phase cerebral thrombosis, pulmonary embolism), transient ischemic attack (TIA), diabetic neuropathy, ischemic disorder (e.g., cerebral infarction, myocardial infarction), angina (e.g., stable angina, unstable angina), chronic kidney diseases including glomerulonephritis and diabetic nephropathy at any stage, allergy, bronchial asthma, restenosis after coronary intervention such as atherectomy and stent implantation, thrombocytopenia by dialysis, the diseases in which fibrosis of organs or tissues is involved [e.g., renal diseases such as tubulointerstitial nephritis), respiratory diseases (e.g., interstitial pneumonia, (idiopathic) pulmonary fibrosis, chronic obstructive pulmonary disease), digestive diseases (e.g., hepatocirrhosis, viral hepatitis, chronic pancreatitis and scirrhous stomachic cancer), cardiovascular diseases (e.g, myocardial fibrosis), bone and articular diseases (e.g, bone marrow fibrosis and rheumatoid arthritis), skin diseases (e.g, cicatrix after operation, scalded cicatrix, keloid, and hypertrophic cicatrix), obstetric diseases (e.g., hysteromyoma), urinary diseases (e.g., prostatic hypertrophy), other diseases (e.g., alzheimer's disease, sclerosing peritonitis, type I diabetes and organ adhesion after operation)], erectile dysfunction (e.g., diabetic erectile dysfunction, psychogenic erectile dysfunction, psychotic erectile dysfunction, erectile dysfunction associated with chronic renal failure, erectile dysfunction after intrapelvic operation for removing prostata, and vascular erectile dysfunction associated with aging and arteriosclerosis), inflammatory bowel disease (e.g., ulcerative colitis, Crohn's disease, intestinal tuberculosis, ischemic colitis and intestinal ulcer associated with Behcet disease), gastritis, gastric ulcer, ischemic ophthalmopathy (e.g., retinal artery occlusion, retinal vein occlusion, ischemic optic neuropathy), sudden hearing loss, avascular necrosis of bone, intestinal damage caused by administration of a non-steroidal anti-inflammatory agent and symptoms associated with lumbar spinal canal stenosis.

Preferably, the pharmaceutical composition according to any one of the preceding embodiments may be used in the prevention or treatment of ulcer, digital ulcer, diabetic gangrene, diabetic foot ulcer, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, or respiratory diseases.

Preferably, the pharmaceutical composition according to any one of the preceding embodiments may be used in the prevention or treatment of pulmonary arterial hypertension (PAH).

It is to be understood that the pharmaceutical composition according to any one of the preceding embodiments may be used for the manufacture of a medicament, in particular for a medicament for preventing and/or treating the above-referenced indications.

It is further to be understood that the present invention also relates to a method for preventing and/or treating the diseases of embodiment 13).

14) A further embodiment of the present invention relates to a process for manufacturing the pharmaceutical composition according to any one of embodiments 1 to 9, comprising the steps of
(a) mixing the compound of formula (I) or a pharmaceutically acceptable salt, solvate, hydrate or morphological form thereof with a filler;
(b) adding a filler and a disintegrant to the blend of step (a) and mixing it;
(c) wet-granulating the blend received from step (b) with a solution comprising the binder;
(d) drying and milling the granulate of step (c);
(e) lubricating the granulate with a lubricant in a suitable blender;
(f) compressing the granulate into core tablets.

Further, the tablet cores are film-coated, dried and polished. Preferably the tablets are film-coated.

The following non-limitative examples illustrate the invention.

### EXAMPLES

Abbreviations (as Used Herein and in the Description Above):

ERA endothelin receptor antagonist

IP receptor Prostacyclin receptor, also termed prostaglandin 12 receptor

mcg microgram

PAH pulmonary arterial hypertension

PDE-5 inhibitor phosphodiesterase type 5 inhibitor

PGI2 Prostaglandin I2

WHO world health organization

1. Preparation of COMPOUND:

The preparation of selexipag (COMPOUND: 2-{4-[N-(5,6-diphenylpyrazin-2-yl)-N-isopropylamino]butyloxy}-N-(methylsulfonyl)acetamide) is described in WO2002/088084. The preparation of polymorphic forms, i.e. the crystalline forms I, II, and III of the free base is disclosed in WO2010/150865; polymorphic forms of pharmaceutically acceptable salts are disclosed in WO2011/024874. COMPOUND was used in the following Examples and assays in form of the free base, especially crystals of polymorphic form I.

2. Quantitative Composition of Selexipag Film-Coated Tablet

TABLE 2
Quantitative composition of selexipag film-coated tablet

| Ingredient | Amount per tablet | |
|---|---|---|
| Dose strength | 100 | μg |
| ACT-293987 | 0.10 | mg |
| D-Mannitol | 9.07 | mg |
| Maize starch | 6.04 | mg |
| Low substituted hydroxypropylcellulose | 0.86 | mg |
| Hydroxypropylcellulose | 0.68 | mg |
| Magnesium stearate | 0.25 | mg |
| Core tablet weight | 17.00 | mg |
| HPMC filmcoat and pigments | 0.63 | mg |
| Carnauba wax | Small quantity | |
| Coating weight | 0.63 | mg |
| Total weight of film-coated tablet | 17.63 | mg |

TABLE 3
Quantitative composition of selexipag film-coated tablet

| Ingredient | Amount per tablet | |
|---|---|---|
| Dose strength | 150 | μg |
| ACT-293987 | 0.90 | mg |
| D-Mannitol | 9.07 | mg |
| Maize starch | 6.04 | mg |
| Low substituted hydroxypropylcellulose | 0.86 | mg |
| Hydroxypropylcellulose | 0.68 | mg |
| Magnesium stearate | 0.25 | mg |
| Core tablet weight | 17.00 | mg |
| HPMC filmcoat and pigments | 0.63 | mg |
| Carnauba wax | Small quantity | |
| Coating weight | 0.63 | mg |
| Total weight of film-coated tablet | 17.63 | mg |

The film-coated tablet shown in Table 2 and 3 is a mini-tablet having a diameter of approximately 3 mm, which makes it easy to swallow for children.

4. Manufacturing Process

i) Mixing

Selexipag and D-mannitol are blended in a suitable blender.

ii) Mixing

Maize starch and low-substituted hydroxypropylcellulose are then added to the blender. The mixture is blended.

iii) Wet Granulation

The blend is transferred into a fluid bed granulator/dryer and a solution of hydroxypropylcellulose in water is sprayed, maintaining the product at a temperature of approximately 30-35° C.

iv) Drying and Milling

The wet granulate is dried in the fluid bed dryer and milled.

v) Lubrication

The granulate is lubricated with magnesium stearate in a suitable blender.

vi) Compression

The final blend is compressed into core tablets.

vii) Coating

The tablet cores are loaded into the pan, and the coating suspension is sprayed until reaching the tablet conformity weight. The tablets are cooled until they are fully dried.

viii) Polishing

The film-coated tablets are polished using carnauba wax.

ix) Packaging

The film-coated tablets are packed in high-density polyethylene bottles with child-resistant polypropylene caps, and containing one desiccant.

1. A pharmaceutical composition comprising the compound of formula (I) in the amount of 80 to 170 mcg 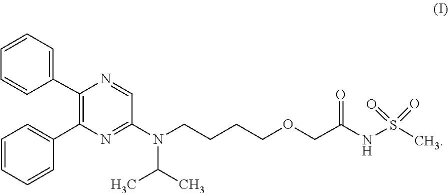 [SMILES: CC(C)N(CCCCOCC(=O)NS(C)(=O)=O)c1cnc(-c2ccccc2)c(-c2ccccc2)n1]
or a pharmaceutically acceptable salt, solvate, hydrate or morphological form thereof.

2. The pharmaceutical composition according to claim 1, further comprising one or more selected from the group consisting of:
a) a filler;
b) a disintegrant;
c) a binder; and
d) a lubricant.

3. The pharmaceutical composition according to claim 2, wherein
the filler, if present, is one or more selected from the group consisting of: D-mannitol, maize starch, lactose, pregelatinized starch, dibasic calcium phosphate dihydrate (CaHPO4.2H2O), microcrystalline cellulose, and maltodextrin;
the disintegrant, if present, is one or more selected from the group consisting of: low substituted hydroxypropyl cellulose, croscarmellose sodium, sodium starch glycolate, and cross-linked polyvinylpyrrolidone;
the binder, if present, is one or more selected from the group consisting of: hydroxypropyl cellulose, sucrose, gelatine, starch, pregelatinized starch, alginic acid, sodium alginate, methyl cellulose, ethyl cellulose, hydroxy propyl methyl cellulose, polyvinyl pyrrolidinone, calcium carboxymethylcellulose, sodium carboxymethylcellulose, guar gum, clays, ion exchange resins and calcium silicate;
the lubricant, if present, is one or more selected from the group consisting of: magnesium stearate, aluminium stearate, calcium stearate, stearic acid, sodium stearyl fumarate, talc, sodium benzoate, glyceryl mono fatty acid, polyethylene glycol, hydrogenated cotton seed oil, castor seed oil, sucrose esters, calcium silicate and silicon dioxide.

4. The pharmaceutical composition according to claim 1, wherein
(i) the filler is comprised in an amount from 11.5 to 145.0 mg;
(ii) the disintegrant is comprised in an amount from 0.6 to 8.5 mg;
(iii) the binder is comprised in an amount from 0.5 to 6.5 mg; and
(iv) the lubricant is comprised in an amount from 0.2 to 2.5 mg.

5. The pharmaceutical composition according to claim 1, wherein
(i) the filler is comprised in an amount from 12.0 to 45.0 mg;
(ii) the disintegrant is comprised in an amount from 0.6 to 2.5 mg;
(iii) the binder is comprised in an amount from 0.5 to 2.0 mg; and
(iv) the lubricant is comprised in an amount from 0.2 to 0.7 mg.

6. The pharmaceutical composition according to claim 1, which comprises
D-mannitol and maize starch;
low substituted hydroxypropyl cellulose;
hydroxypropyl cellulose; and
magnesium stearate.

7. The pharmaceutical composition according to claim 6, which comprises
D-mannitol in an amount from 7.0 to 90.0 mg;
maize starch in an amount from 4.5 to 60.0 mg;
low substituted hydroxypropyl cellulose in an amount from 0.6 to 9.0 mg;
hydroxypropyl cellulose in an amount from 0.5 to 6.5 mg; and
magnesium stearate is comprised in an amount from 0.2 to 2.5 mg.

8. The pharmaceutical composition according to claim 6, which comprises
D-mannitol in an amount from 7.0 to 25.0 mg;
maize starch in an amount from 4.5 to 20.0 mg;
low substituted hydroxypropyl cellulose in an amount from 0.6 to 3.0 mg;
hydroxypropyl cellulose in an amount from 0.5 to 2.0 mg; and
magnesium stearate is comprised in an amount from 0.2 to 0.7 mg.

9. The pharmaceutical composition according to claim 1, which is in the form of a tablet.

10. The pharmaceutical composition according to claim 9, wherein the tablet is coated, the coating material comprising one or more selected from the group consisting of a plasticizer, a film former and a pigment.

11. The pharmaceutical composition according to claim 9, wherein the tablet is coated, the coating material comprising one or more selected from the group consisting of a plasticizer, a film former, a glidant and a pigment.

12. The pharmaceutical composition according to claim 9, wherein the tablet has a diameter of 1.5 to 4 mm.

13. (canceled)

14. The method according to claim 17, where the patient is a pediatric patient.

15. A method for treating a patient with hepatic impairment or a patient experiencing drug drug interaction with a CYP 2C8 inhibitor, comprising administering a pharmaceutical composition of the claim 1 to the patient.

16. (canceled)

17. A method treating or preventing pulmonary arterial hypertension (PAH) in a subject in need thereof, comprising administering a pharmaceutical composition of the claim 1 to the subject.

18. A method for preventing and/or treating ulcer, digital ulcer, diabetic gangrene, diabetic foot ulcer, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance, connective tissue disease, chronic kidney diseases, diseases in which fibrosis of organs or tissues is involved, or respiratory diseases in a human subject in need thereof, comprising administering the pharmaceutical composition according to claim 1 to the human subject in need thereof.

19. The method according to claim 18, wherein the human subject is from ≥2 years to <18 years old.

20. The method according to claim 18, wherein the human subject is a patient with hepatic impairment or a patient experiencing drug drug interaction with CYP 2C8 inhibitors.

21. A process for manufacturing the pharmaceutical composition according to claim 1, comprising the steps of
a) mixing the compound of formula (I) or a pharmaceutically acceptable salt, solvate, hydrate or morphological form thereof with a filler;
b) adding a filler and a disintegrant to the blend of step a) and mixing it;
c) wet-granulating the blend received from step (b) with a solution comprising the binder;
d) drying and milling the granulate of step (c);
e) lubricating the granulate with a lubricant in a suitable blender; and
f) compressing the granulate into core tablets.

22. The method according to claim 18, wherein the chronic kidney disease is glomerulonephritis or diabetic nephropathy at any stage
